(19) United States
(12) Patent Application Publication
(10) Pub. No.: US 2023/0263793 A1
(43) Pub. Date: Aug. 24, 2023

(54) PHARMACEUTICAL COMPOSITION FOR PREVENTING OR TREATING VIRAL INFECTIONS

(71) Applicants: Tomas Lluis Manent, Barcelona (ES); Eric François Faucillon, Vannes (FR)

(72) Inventors: Tomas Lluis Manent, Barcelona (ES); Eric François Faucillon, Vannes (FR)

(21) Appl. No.: 18/019,684

(22) PCT Filed: Aug. 13, 2021
(86) PCT No.: PCT/IB2021/057466

§ 371 Date: Feb. 3, 2023

(30) Foreign Application Priority Data
Aug. 21, 2020 (GB) 2013131.4
Jan. 19, 2021 (GB) 2100711.7

(51) Int. Cl. A61K 31/465; A61K 9/00; A61K 31/444; A61K 31/198; A61K 31/12; A61K 31/443

(52) U.S. Cl. CPC A61K 31/465; A61K 9/0056; A61K 31/444; A61K 31/198; A61K 9/0043; A61K 9/0048; A61K 31/12; A61K 31/443

(57) ABSTRACT

A pharmaceutical composition containing specific nicotine-related alkaloids in a nasal formulation, or in combination with other compounds from tobacco and/or natural plant species, in nasal, oral or ocular formulations, for use as an effective treatment for viral infections including Covid-19. Novel compositions comprise one or more of the compounds nicotine, nornicotine, anabasine, anabaseine, anatabine, cotinine, myosmine, N-formyl-nicotine, isonicotine, and nicotyrine; and one or more of the compounds S-allyl-cysteine, S-allyl mercaptocysteine, 6-shogaol, 6 gingerol, curcumin and piperine; wherein the composition is in the form of a liquid or powdered nasal, liquid oral, or liquid ocular formulation, except that neither curcumin or piperine are not present in a nasal or ocular formulation.

This invention relates to a pharmaceutical composition and in particular to the use of a pharmaceutical composition containing nicotine-related alkaloids for treating antiviral infections. The invention also relates to novel compositions.

A viral infection that is a global concern is Covid-19, which is an infectious disease caused by the coronavirus SARS-CoV-2. This virus and disease have caused a serious pandemic, with hundreds of thousands of deaths globally. There is a need for an effective antiviral to treat Covid-19.

The present invention tackles this problem by the use of a particular formulations and/or combinations of compounds from natural sources. Natural products, including plant species, provide a huge diversity of chemical complexity; many pharmaceutical products which have been developed in the past are derived from natural products. Often the challenge has been that the individual constituent compounds isolated from a natural source are less active than the original natural material, either because of the route of administration or because of synergistic mechanisms between the compounds within a plant or between different plants.

The present invention meets this challenge by identifying particular formulations and/or synergistic combinations. In particular, the present invention relates to a pharmaceutical composition containing specific nicotine-related alkaloids in a nasal formulation, or in combination with other compounds from tobacco and/or natural plant species, in nasal, oral or ocular formulations, for use as an effective treatment for viral infections including Covid-19.

Formulations of nicotine and related tobacco alkaloids have been used therapeutic compositions, and in nicotine replacement therapy to help smokers give up smoking.

Some literature has suggested the use of nicotine as an antiviral.

Kloc et al (Immunology Letters vol 224, 2020, 38-29) proposes that nicotine may prevent or lessen the severity of Covid-19, based on surveys showing less severe symptoms in Covid-19 patients who smoke.

Changeux et al (C R Biol. 2020 Jun 5; 343(1): 33-39) suggests that the nicotinic acetylcholine receptor (nAChR) may play a key role in the pathophysiology of SARS-CoV-2 infection; and suggests that nicotine may be a potential preventive agent against Covid-19 infection.

Tizabi et al, (FEBS Journal, vol 287, 2020) describes a therapeutic role for nicotine in the treatment of Covid-19.

Farsalinos et al, (International Journal of Molecular Sciences, 2020, 21, 5807) provides a hypothesis that nicotine and other cholinergic agonists varenicline and galantamine may have value as therapeutic interventions for Covid-19.

Farsalinos et al, (Toxicology Reports, Volume 7, 2020, Pages 658-663), also hypothesizes that Covid-19 may be a disease of the nicotinic cholinergic system and that nicotine could maintain or restore the function of the cholinergic anti-inflammatory system.

US patent application 2020/179367 relates to a method of treating a coronavirus comprising administering isomyosmine, which is a constituent of tobacco.

However, none of those documents disclose, or suggest, the use of a nasal formulation of tobacco alkaloids that form part of the present invention. Furthermore, none of those documents disclose, or suggest, a synergistic combination of tobacco alkaloids to enhance antiviral activity.

Accordingly, the present invention provides a pharmaceutical composition for use in the prevention or treatment of a viral infection, which composition comprises, as an active ingredient or ingredients, one compound selected from nornicotine, anabasine, anabaseine, anatabine, cotinine, myosmine, N-formyl-nicotine, isonicotine, and nicotyrine; or two or more compounds selected from nicotine, nornicotine, anabasine, anabaseine, anatabine, cotinine, myosmine, N-formyl-nicotine, isonicotine, and nicotyrine; wherein the composition comprising a single active ingredient is in the form of a liquid or powdered nasal formulation; and the composition comprising two or more active ingredients is in the form of a liquid or powdered nasal, liquid oral, or liquid ocular formulation.

Preferably, the composition also comprises a pharmaceutically acceptable carrier.

Thus, one embodiment of the present invention provides a pharmaceutical composition for use in the prevention or treatment of a viral infection, which composition comprises, as an active ingredient, one compound selected from nornicotine, anabasine, anabaseine, anatabine, cotinine, myosmine, N-formyl-nicotine, isonicotine, and nicotyrine; wherein the composition is in the form of a liquid or powdered nasal formulation.

In a further embodiment, the present invention provides a pharmaceutical composition for use in the prevention or treatment of a viral infection, which composition comprises, as active ingredients, two or more of the compounds nicotine, nornicotine, anabasine, anabaseine, anatabine, cotinine, myosmine, N-formyl-nicotine, isonicotine, and nicotyrine; wherein the composition is in the form of a liquid or powdered nasal, liquid oral, or liquid ocular formulation.

Suitable liquid nasal formulations for use in this invention include nasal drops, nasal sprays, nasal nebulizers, aerosols, and nasal inhalation solutions.

Suitable liquid oral formulations for use in this invention include oral drops, oral sprays, oral nebulizers, aerosols, oral inhalation solutions, liquid capsules, lozenges, liquids, syrups, elixirs, and suspensions.

Suitable liquid ocular formulations for use in this invention include eye drops.

Suitable nasal powdered formulations for use in this invention include dry extract and insufflation powders.

Preferred forms of the compositions for use in this invention having two or more active ingredients are nasal drops, nasal sprays, nasal nebulizers, nasal inhalation solutions, oral inhalation solutions for oral inhalers, oral drops, aerosols, and eye drops. Preferred such formulations are a nasal spray or oral drops, especially a nasal spray.

For example, the compositions for use in this invention having two or more active ingredients may be administered in an oral or nasal inhaler, a nasal spray, a pressurized aerosol, nasal drops, or eye drops. The composition may be nasally administered with a nasal spray device to deliver a mist spray to nasal mucosa, for example a spray-squeeze bottle or a pump vaporizer

The compounds nicotine, nornicotine, anabasine, anabaseine, anatabine, cotinine, myosmine, N-formyl-nicotine, isonicotine, and nicotyrine are alkaloids that are present in species of the plant genus Nicotiana.

Nicotiana is a genus of herbaceous plants, commonly known as tobacco plants. Species of Nicotiana that may provide a source of compounds in the compositions for use in this invention include, for example, N. acuminate, N. Africana, N. alata, N. attenuata, N. benthamiana, N. clevelandii, N. glauca, N. glutinosa, N. langsdorffii, N. longiflora, N. occidentalis, N. obtusifolia, N. otophora, N. plumbaginifolia, N. quadrivalvis, N. rustica, N. suaveolens, N. sylvestris, N. tabacum, N. tomentosiformis.

The most widely used species is N. tabacum, which is grown worldwide for production of tobacco products including cigarettes.

The constituents of the composition for use in this invention may include nicotine with other tobacco alkaloids. Nevertheless, the composition and administration routes of this invention do not have the toxicity and addictive properties associated with tobacco products, since this composition does not have toxic and/or addictive additives, usually present in cigarettes. Furthermore, because the compositions provide an antiviral medicinal product, there is no social behaviour component linked to psychological aspects of addiction.

Although smoking tobacco is generally regarded to be carcinogenic, in fact the toxic effects caused by cigarette smoking are not primarily due to nicotine, but instead the carcinogens and other toxins produced by the burning and inhalation of tobacco. Nicotine itself is not carcinogenic. Other known routes of administration of nicotine, free from and chemical additives that are not present in the tobacco plant, have proven therapeutic effects in multiple pathologies.

Furthermore, the degree of addiction of natural nicotine can be equated to that of caffeine. Wild tobacco itself does not contain toxins that induce or enhance addictive effects, which are present in industrial cigarettes.

The compositions for use in this invention may contain two or more tobacco alkaloids. In this way, each constituent acts in synergy with the others as an adjuvant or modulator, to enhance the effect of the composition against a viral disease or its complications such as cardiovascular damage, neurocognitive disorders, or neuronal degeneration.

Although this aspect of the invention is not limited to a particular mechanism, it is believed that the combination of the tobacco alkaloids ameliorates the cytokine storm and severe related inflammatory response through an alpha-7 nicotinic acetylcholine receptor (nAChR)-mediated cholinergic anti-inflammatory pathway.

Nicotine is 3-(1-methyl-2-pyrrolidinyl)pyridine. Nicotine is an exogenous alpha-7-nAchR agonist, and is clinically used in ulcerative colitis to counteract inflammation. With respect to the coronavirus SARS-CoV-2, nicotine may inhibit or have some direct or indirect antiviral effect on ACE2/SARS-CoV-2-S complex. In addition, nicotine has a positive effect on the vagus nerve regulation, whose function is downregulated and disrupted in some Covid-19 patients.

Nornicotine is 3-pyrrolidin-2-ylpyridine. In the compositions for use in the present invention, nornicotine can either substitute for nicotine and evoke midbrain dopamine release; or contribute to the neuropharmacological effects of nicotine.

Anabasine is 3-piperidin-2-ylpyridine. It increases the effect of the other tobacco alkaloids, by acting synergistically; and independently acts in the regulation of the alpha-7-nicotinic acetylcholine receptors. Anabasine has a significantly increased life span compared to nicotine. It is also a potent anti-inflammatory agent and transynaptic facilitator at low doses.

Anabaseine is 3,4,5,6-Tetrahydro-2,3′-bipyridine. The compound is a potent nicotinic receptor agonist and stimulates a wide variety of animal nicotinic acetylcholine receptors (AChRs). Anabaseine, separately or in synergistic effect with other tobacco alkaloids, can improve cognitive disorders observed in patients.

Anatabine is 3-[(2S)-1,2,3,6-tetrahydropyridin-2-yl] pyridine. It has a synergistic effect on the locomotor activity of other alkaloids including nicotine. Anatabine also has specific individual affinity for nAChRs.

Cotinine is (5S)-1-methyl-5-(3-pyridyl) pyrrolidin-2-one. Cotinine is a positive modulator of the alpha 7 nicotinic acetylcholine receptor. It has a longer average life than nicotine and therefore increases the duration of the nicotine's activity. It can act as a cognitive enhancer and neuroprotector both therapeutically and prophylactically.

Myosmine is 3-(3,4-Dihydro-2H-pyrrol-5-yl) pyridine. It enhances nicotine's effects on locomotor activity and acts as an allosteric modulator at nicotinic acetylcholine receptors, producing partial desensitization of the receptors.

Other tobacco alkaloids which may be present in the compositions for use in this invention are:

N-formyl-nornicotine;

Isonicotine, which is 4-(4-piperidinyl)pyridine; and

Nicotyrine, which is 3-(1-methylpyrrol-2-yl)pyridine.

The compositions for use in this invention preferably contain two, three, four, five or six constituents selected from nornicotine, anabasine, anatabine, cotinine and myosmine. A preferred constituent is nicotine. For example, the compositions may contain nicotine and either anabasine, or anatabine. A preferred form of those compositions is a nasal spray.

Suitably, the compositions contain nicotine plus four compounds selected from nornicotine, anabasine, anatabine, cotinine and myosmine. Each constituent enhances the effect of nicotine in a different manner; thus, preferably the composition for use in this invention comprises all of the compounds: nicotine, nornicotine, anabasine, anatabine, cotinine and myosmine.

In a further embodiment of this invention, novel compositions are provided by the incorporation of additional compounds derived from other plant sources, depending on the route of administration.

Therefore, the present invention also provides a pharmaceutical composition comprising one or more, preferably two or more, of the compounds nicotine, nornicotine, anabasine, anabaseine, anatabine, cotinine, myosmine, N-formyl-nicotine, isonicotine, and nicotyrine; and one or more of the compounds S-allylcysteine, S-allyl mercaptocysteine, 6-shogaol, and 6-gingerol; wherein the composition is in the form of a liquid or powdered nasal, liquid oral, or liquid ocular formulation.

In a further embodiment, the present invention provides a pharmaceutical composition comprising one or more, preferably two or more, of the compounds nicotine, nornicotine, anabasine, anabaseine, anatabine, cotinine, myosmine, N-formyl-nicotine, isonicotine, and nicotyrine; and one or more of the compounds S-allylcysteine, S-allyl mercaptocysteine, 6-shogaol, 6-gingerol, curcumin, and piperine; wherein the composition is in the form of a liquid oral formulation.

Preferred such formulations contain S-allylcysteine, S-allyl mercaptocysteine, 6-shogaol, and 6-gingerol.

The compounds S-allylcysteine (SAC), and S-allyl mercaptocysteine (SAMC) are present in Allium sativum, which is commonly known as garlic. Garlic is well known for human consumption and use, especially as a common seasoning worldwide. These compounds act in synergy with the tobacco alkaloids to protect tissues against oxidative stress.

S-Allylcysteine has antioxidative properties, cardiovascular protection, and neuronal degeneration protection effects. It can enhance anti-inflammatory, antiviral, immunomodulation and neuroprotective effects of other tobacco alkaloids. In Covid-19 patients, it would protect the heart and brain against damage.

The compounds 6-shogaol and 6-gingerol, are present in Zingiber officinale, which is a flowering plant whose rhizome, ginger, is widely used as a spice and a folk medicine. These compounds act in synergy with the tobacco alkaloids to diminish the production of inflammatory cytokines observed in severe and critical Covid-19 patients; support enterocites regulation; and provide neuroprotection.

The compound curcumin is present in Curcuma longa, which is known commonly as turmeric, a flowering plant of the ginger family, Zingiberaceae, the roots of which are used in cooking. Curcumin acts in synergy to effect the metabolic inactivation of the amino acid sequence used by SARS-COV-2 S glycoprotein in its binding to ACE2 human enzyme. Curcumin also exhibits properties in regulating the anti-inflammatory cholinergic pathway disrupted in Covid-19 disease.

The compound piperine is present in Piper nigrum, which is a flowering vine in the family Piperaceae, cultivated for its fruit, known as a peppercorn, which is usually dried and used as a spice and seasoning. Piperine acts in synergy to protect against Covid-19 acute lung injury.

Because of lever clearance diminution, piperine is able to significantly increase the bioavailability of curcumin.

One suitable composition of this invention comprises a combination of nicotine, S-allylcysteine, and one or more compounds selected from nornicotine, anabasine, anabaseine, anatabine, cotinine, myosmine, N-formyl-nicotine, isonicotine, and nicotyrine, preferably in the form of a nasal spray.

In a particular embodiment, the composition comprises a combination of S-allylcysteine with one or more compounds selected from nicotine, anabasine, anatabine, and cotinine.

For example, the composition may a combination of S-allylcysteine, nicotine and either or both anatabine and anabasine.

Preferred compositions of this invention comprise combinations of:

nicotine, anabasine and S-allylcysteine; or

nicotine, anatabine and S-allylcysteine; or

cotinine, anabasine and S-allylcysteine.

The compositions of this invention may also comprise additional constituents that are cholinergic agonists, such as epibatidine, cysteine, pilocarpine, carbachol, bethanechol, methacholine, or acetylcholine.

The major constituent of the compositions for use in the present invention may be nicotine, nornicotine, anabasine, anabaseine, anatabine, cotinine, myosmine, N-formyl-nornicotine; isonicotine, or nicotyrine.

The major constituent may be suitably from 50% to 95% by weight of the constituents. Preferably the major constituent comprises from 70% to 98%, for example from 80 to 95% by weight of the constituents

Alternatively, any of the compounds may be minor constituents of the compositions. In that case, each minor constituent may comprise from 0.2% to 26%, suitably from 1% up to 16%, preferably from 6% to 11%, for example from 7% to 10% by weight of the constituents.

If S-allylcysteine is present in the constituents, it may comprise from 0.001% to 0.1%, suitably from 0.005% up to 0.05%, preferably from 0.009% to 0.04%, for example from 0.01% to 0.04% by weight of the constituents.

The compositions of the invention may further comprise a pharmaceutically acceptable carrier. The carrier may include one or more excipients or diluents, such as solvents, dispersion media, coatings, isotonic and absorption delaying agents, binders, lubricants, disintegrants, colouring agents, bulking agents, flavouring agents, sweetening agents, and buffers. Examples of such excipients include gelatin, gum arabicum, lactose, microcrystalline cellulose, starch, sodium starch glycolate, calcium hydrogen phosphate, magnesium stearate, talcum, and colloidal silicon dioxide.

The pharmaceutically acceptable carrier may be a liquid. Liquid carriers are used in preparing solutions, suspensions, emulsions, syrups, elixirs, drops, sprays, and pressurized compositions. Examples of liquid carriers are water, an organic solvent, a mixture of both, or a pharmaceutically acceptable oil or fat.

A preferred excipient for oral, nasal, or ocular formulations is sterile water, optionally with an alcohol, such as ethanol.

The compositions of this invention may also contain a preservative and/or stabilizer. These include, for example: ethylene diamine tetra-acetic acid, lower alkyl p-hydroxybenzoates, chlorhexidine, quaternary ammonium compounds, for example cetylpyridinium chloride, tetradecyltrimethyl ammonium bromide, and myristyl picolinium chloride. Each of these compounds may be used in a concentration of 0.002 to 0.05%, for example 0.02% (weight/volume in liquid compositions, weight/weight in solid compositions).

The compositions of this invention may also be formulated by encapsulation in a colloidal drug delivery system. Suitable colloidal drug delivery systems include lipid-based and polymer-based colloidal drug delivery systems such as liposomes, nanoparticles, microparticles or block copolymer micelles.

Alternatively, the compositions may be encapsulated in a polymeric, non-colloidal drug delivery system, such as hydrogels and films.

The compositions of the invention may also be formulated as nanoparticles, for example with a mean particle size of 0.13 μm. The mean particle size of normal powder is 22.87 μm.

The compositions of this invention are prepared by mixing the dry constituents and incorporating into the desired liquid or solid formulation.

The constituents of the compositions are known compounds, which are commercially available, or may be synthesised by known methods.

Alternatively, the compounds may be isolated from extracts of species of Nicotiana, or the other plant materials that contain the compounds, as specified herein.

In the nasal spray or eye drop form of the composition of the present invention, suitably 0.005 to 1.3 ml of the liquid composition may be released per individual application, preferably from 0.01 to 0.25 ml per application, for example from 0.05 to 0.25 ml per application. The amount of active ingredient delivered from a nasal spray or eye drop formulation per application may be suitably from 0.035 mg to 9 mg, preferably from 0.07 mg to 1.73 mg, for example from 0.35 to 1.73 mg of active ingredient.

In the solid powder nasal inhalation form of the composition of the present invention, suitably from 0.035 mg up to 9 mg of the active compounds may be released per individual application, preferably from 0.07 mg to 1.73 mg per application, for example, 0.35 mg to 1.73 mg of the active ingredients. In addition to these active compounds, a suitable inactive powder containing excipients and vehicle may be added.

The compositions of this invention can be administered to a mammal, including human, for the prevention or treatment of a disease caused by the coronavirus SARS-CoV-2, in particular Covid-19.

The compositions are particularly useful for human treatment.

The viral disease may also include diseases within the coronaviruses family, such as, for example, SARS-Cov-1 and the common cold. The viral disease may also include other viral infections that are not of coronavirus origin, but that dysregulate the cholinergic system, for example the influenza virus.

In addition, the viral disease may be any mutation or alternative strain of the SARS-CoV-2 virus.

The compositions of this invention are not only antiviral agents, but also anti-inflammatory and immunomodulatory agents.

Although the invention is not limited to a particular mechanism, it is believed that the compositions exhibit a direct antiviral action both by antagonizing the binding of the ACE-2 Spike protein and also by antagonizing the disruption of the nicotinic acetylcholine receptor (nAChR). Such a combination of antiviral actions means that the compositions act both in a protective, i.e. prophylactic, manner, as well as a direct therapeutic manner.

Furthermore, the compositions of the invention protect and regulate the Renin-Angiotensin-Aldosterone System (RAAS), which regulates kidney, lung, heart, intestinal and brain functions.

The unique combination of activities results in the compositions of this invention being useful for treating a further related condition. Persistent chronic fatigue symptoms have been frequently observed in survivors of viral infections, including Severe Acute Respiratory Syndrome (SARS). This has been observed in the case of SARS-Cov-1, in 2003, and more recently in SARS-CoV-2. For example, in COVID-19 infected patients that have overcome the disease, structural viral damage of organs may remain for a prolonged time. Multiorgan symptoms following an infection are being reported by increasing numbers of patients; the condition is known as Post-Covid-19 syndrome or ‘Long-Covid’. The symptoms include cough, breathlessness, chronic fatigue, headache, chest pain, joint pain, insomnia, anxiety and stress. It has been found that the compositions of this invention speed the recovery of patients suffering from such symptoms.

The compositions of this invention can therefore be administered to a mammal, including human, for the prevention or treatment of symptoms following a viral infection, in particular a SARS infection, such as Post-Covid-19 syndrome.

The dosages of the compositions of this invention are adjusted according to the form of administration. For example, a nasal spray formulation is more effective than an oral liquid formulation, so that smaller dose of the constituents by weight is required when applied via a nasal spray than via oral liquids.

If the administration route is the oral liquid form of the compositions of this invention, the dose of the active ingredients of this invention may be, for example, from 0.7 mg to 110 mg per day, suitably from 2.7 mg to 28 mg per day, preferably from 11 mg to 14 mg per day. The total daily dosage is conveniently administered in three portions of from 0.1 ml to 16 ml, suitably from 0.4 ml to 4 ml, preferably from 1.6 ml to 2 ml of the oral liquid formulation.

If the administration route is the nasal spray or eye drop form of the compositions of this invention, the dose of the active ingredients of this invention may be, for example, from 0.07 mg to 18 mg, preferably from 0.14 mg to 3.5 mg, for example from 0.7 mg to 3.5 mg per day. For a nasal spray, the total daily dosage is conveniently administered in a single spray application in each nostril per day. For eye drops, the total daily dosage is conveniently administered in a single eye drop application in each eye per day.

If the administration route is the nasal powder form of the compositions of this invention, the dose of the active ingredients may be suitably from 0.07 mg up to 18 mg of the active compounds per day, preferably from 0.14 mg to 3.4 mg per day, for example, 0.7 mg to 3.4 mg per day. The total daily dosage is conveniently administered in a single nasal powder application in each nostril per day.

The dosages stated above are suitable for non-smoking adults as a prophylactic, or for health personnel at risk of contagion, or for patients with mild symptoms of the virus, including SARS-CoV-2.

For patients with moderate to severe symptoms, especially if hospitalized, the daily dosage of an oral liquid formulation could be increased by 10% to 20% from the figures for prophylactic or mild symptoms; and the daily dosage of nasal spray could be doubled, suitably by administering two actuations per nostril per day, instead of one; the daily dosage of nasal powder formulation could be doubled, suitably by administering two actuations per nostril per day, instead of one; and the daily dosage of eye drops spray could be doubled, suitably by administering two applications per eye per day, instead of one.

For patients with severe symptoms, especially those requiring intubation, the daily dosage of an oral liquid formulation could be increased by 30% to 40% from the figures for prophylactic or mild symptoms; and the daily dosage of nasal spray could be trebled, suitably by administering three actuations per nostril per day, instead of one; the daily dosage of nasal powder formulation could be trebled, suitably by administering three actuations per nostril per day, instead of one; and the daily dosage of eye drops could be trebled, suitably by administering three applications per eye per day, instead of one.

For non-smoking patients, it is also advantageous to administer eye drops or an oral liquid form prior to the use of a nasal spray. This initial application of a nicotine formulation prepares the body for the administration of a rapidly absorbed amount of nicotine alkaloid, and so prevents any adverse reaction thereto.

For patients who smoke, the dosages of an oral liquid formulation may be increased by 20% to 70%; and the dosage of a nasal formulation, eyedrops, and nasal powder could be augmented up to 4 times from the daily basis dose, depending on the level of smoking. Because females have lower serum ACE2 levels than males, the dosages for females may be 70% of the dosages for males.

The present invention is illustrated by the following examples.

### EXAMPLE 1

### Oral Drops

Oral drops were prepared by direct purification and isolation through high-performance liquid chromatography (HPLC) of the natural products in the following amounts:

5.64 mg/ml nicotine (81.68% by weight)

20 1.03 mg/ml anabasine (14.9% by weight)

0.23 mg/ml cotinine (3.4% by weight)

0.0014 mg/ml S-allylcysteine (0.02% by weight)

The resulting oral drop solution contained a total of 6.9 mg/ml of the active ingredients.

### EXAMPLE 2

### Oral Drops

Oral drops were prepared by direct purification and isolation through HPLC of the natural products in the following amounts:

5.29 mg/ml cotinine (76.68% by weight)

1.61 mg/ml anabasine (23.3% by weight)

0.0014 mg/ml S-allylcysteine (0.02% by weight)

The resulting oral drop solution contained a total of 6.9 mg/ml of the active ingredients.

### EXAMPLE 3

### Liquid Spray With Nicotine and Anabasine

A liquid spray was prepared by direct purification and isolation through HPLC of the natural products in the following amounts:

5.87 mg/ml nicotine (85.08% by weight)

1.03 mg/ml anabasine (14.92% by weight)

The resulting liquid spray solution contained a total of 6.9 mg/ml of the active ingredients.

This solution may be used as a nasal spray, or an oral inhalation spray.

### EXAMPLE 4

### Liquid Spray With Nicotine, Anabasine and S-allylcysteine

A liquid spray was prepared by direct purification and isolation through HPLC of the natural products in the following amounts:

5.87 mg/ml nicotine (85.05% by weight)

1.03 mg/ml anabasine (14.9% by weight)

0.0014 mg/ml S-allylcysteine (0.02% by weight)

The resulting liquid spray solution contained a total of 6.9 mg/ml of the active ingredients.

This solution may be used as a nasal spray, or an oral inhalation spray.

### EXAMPLE 5

### Liquid Eye Drops With Nicotine, Anabasine and S-allylcysteine

Eye drops were prepared by direct purification and isolation through HPLC of the natural products in the following amounts:

5.87 mg/ml nicotine (89.98% by weight)

1.03 mg/ml anabasine (5% by weight)

0.0014 mg/ml S-Allylcysteine (0.02% by weight)

The resulting liquid eye drops solution contained a total of 6.9 mg/ml of the active ingredients.

### EXAMPLE 6

### Liquid Eye Drops With Nicotine, Anatabine and S-allylcysteine

A liquid eye drops preparation was prepared by direct purification and isolation through HPLC of the natural products in the following amounts: 6.67 mg/ml nicotine (96.58% by weight) 0.23 mg/ml anatabine (3.4% by weight) 0.0014 mg/ml S-Allylcysteine (0.02% by weight)

The resulting liquid eye drop solution contained a total of 6.9 mg/ml of the active ingredients.

1. A pharmaceutical composition for use in the prevention or treatment of a viral infection, which composition comprises, as an active ingredient or ingredients, one compound selected from nornicotine, anabasine, anabaseine, anatabine, cotinine, myosmine, N-formyl-nicotine, isonicotine, and nicotyrine; or two or more compounds selected from nicotine, nornicotine, anabasine, anabaseine, anatabine, cotinine, myosmine, N-formyl-nicotine, isonicotine, and nicotyrine; wherein the composition comprising a single active ingredient is in the form of a liquid or powdered nasal formulation; and the composition comprising two or more active ingredients is in the form of a liquid or powdered nasal, liquid oral, or liquid ocular formulation.

2. The composition as claimed in claim 1, which composition comprises, as an active ingredient, one compound selected from nornicotine, anabasine, anabaseine, anatabine, cotinine, myosmine, N-formyl-nicotine, isonicotine, and nicotyrine; wherein the composition is in the form of a liquid or powdered nasal formulation.

3. The composition as claimed in claim 1, which composition comprises, as active ingredients, two or more of the compounds nicotine, nornicotine, anabasine, anabaseine, anatabine, cotinine, myosmine, N-formyl-nicotine, isonicotine, and nicotyrine;
wherein the composition is in the form of a liquid or powdered nasal, liquid oral, or liquid ocular formulation.

4. The composition as claimed in claim 3, which comprises a combination of nicotine and either anabasine, or anatabine.

5. The composition as claimed in claim 1, to wherein the viral infection is a disease caused by the coronavirus SARS-CoV-2.

6. The composition as claimed in claim 5, for use in the prevention or treatment of Covid-19.

7. A pharmaceutical composition comprising one or more of the compounds nicotine, nornicotine, anabasine, anabaseine, anatabine, cotinine, myosmine, N-formyl-nicotine, isonicotine, and nicotyrine; and one or more of the compounds S-allylcysteine, S-allyl mercaptocysteine, 6-shogaol, 6-gingerol, and curcumin; wherein the composition is in the form of a liquid or powdered nasal, liquid oral, or liquid ocular formulation.

8. A pharmaceutical composition comprising one or more of the compounds nicotine, nornicotine, anabasine, anabaseine, anatabine, cotinine, myosmine, N-formyl-nicotine, isonicotine, and nicotyrine; and one or more of the compounds S-allylcysteine, S-allyl mercaptocysteine, 6-shogaol, 6-gingerol, curcumin, and piperine; wherein the composition is in the form of a liquid oral formulation.

9. The composition as claimed in claim 7 which comprises a combination of S-allylcysteine with one or more compounds selected from nicotine, anabasine, anatabine, and cotinine.

10. The composition as claimed in claim 9 which comprises a combination of S-allylcysteine, nicotine and either or both anatabine and anabasine.

11. The composition as claimed in claim 9 which comprises nicotine, anabasine and S-allylcysteine.

12. The composition as claimed in claim 9 which comprises nicotine, anatabine and S-allylcysteine.

13. The composition as claimed in claim 9 which comprises cotinine, anabasine and S-allylcysteine.

14. The composition as claimed in claim 1, which further comprises one or more cholinergic agonists, selected from epibatidine, cysteine, pilocarpine, carbachol, bethanechol, methacholine, and acetylcholine.

15. The composition as claimed in claim 3 in the form of oral drops, eye drops or nasal spray.

16. The composition as claimed in claim 15 in the form of a nasal spray.

17. A method of prevention or treatment of a viral infection in mammals, including humans, which comprises administering to a mammal a composition comprising two or more of the compounds nicotine, nornicotine, anabasine, anabaseine, anatabine, cotinine, myosmine, N-formyl-nicotine, isonicotine, and nicotyrine; wherein the composition is in the form of a liquid or powdered nasal, liquid oral, or liquid ocular formulation.

18. The method as claimed in claim 17 for the prevention or treatment of a disease caused by the coronavirus SARS-CoV-2.

19. The method as claimed in claim 18 for the prevention or treatment of Covid-19.

20. The method as claimed in claim 17 wherein the composition is administered in the form of a nasal spray
